(19) United States
(12) Patent Application Publication
(10) Pub. No.: US 2021/0069419 A1
(43) Pub. Date: Mar. 11, 2021

(54) CAP AND CARTRIDGE ASSEMBLY

(71) Applicant: NORDSON CORPORATION, Westlake, OH (US)

(72) Inventor: Matthew E. Pappalardo, Ewing, NJ (US)

(21) Appl. No.: 16/981,357

(22) PCT Filed: Mar. 29, 2019
(86) PCT No.: PCT/US2019/024819

§ 371 Date: Sep. 16, 2020

Related U.S. Application Data
(60) Provisional application No. 62/650,215, filed on Mar. 29, 2018.

(51) Int. Cl. A61M 5/31

(52) U.S. Cl. CPC A61M 5/31; A61M 2005/3118; A61M 2005/3106; A61M 2005/3104

(57) ABSTRACT

A fluid cartridge assembly includes a fluid cartridge and a cap. The fluid cartridge includes a cartridge body and a flange extending from the cartridge body. The cap includes a locking latch, first and second squeeze handles, and a lever. The locking latch is configured to engage the flange of the fluid cartridge to couple the cap to the cartridge. The first squeeze handle being operatively coupled to said locking latch and configured to be depressed in a radially inward direction toward said second squeeze handle causing said locking latch to move in a radially outward direction. The lever is coupled to at least one of the first and second squeeze handles and configured to move between two positions. In one position the squeeze handles are substantially prevented from moving in the radially inward direction, and in another position the squeeze handles can move freely in the radially inward direction.

### CROSS REFERENCE TO RELATED APPLICATIONS

This application is a U.S. National Stage Application of International Patent Application No. PCT/US2019/024819, filed Mar. 29, 2019, which claims the benefit of U.S. Provisional Patent App. No. 62/650,215, filed Mar. 29, 2018, the disclosures of both of which are hereby incorporated by reference as if set forth in their entireties herein.

### TECHNICAL FIELD

This disclosure relates generally to a fluid cartridge assembly, and more particularly, to an assembly for sealing a fluid cartridge with a sealing cap.

### BACKGROUND

Sealing a fluid cartridge, such as a syringe, with a sealing cap is generally known in the art. There are different types of fluid cartridge assemblies that include a cap capable of being attached, secured, and removed to and from a fluid cartridge to control fluid from entering and exiting the fluid cartridge.

Examples of sealing cap and cartridge assemblies include twist type connections, barb type connections, snap type connections, or other connections. Twist type connections may include, for example, a threaded connection or a bayonet twist connection between the sealing cap and the fluid cartridge. In twist type connections, the cap can be rotated in opposing directions to attach and to remove the cap to and from the fluid cartridge. With barb type and snap type connections, barbs of the cap attach to flanges of the cartridge. In these conventional cap and cartridge assemblies, sealing caps can be inadvertently removed from the fluid cartridge, which can prematurely leak contents of the fluid cartridge and cause harm to the user or other individuals in proximity to the cap and cartridge assembly.

### SUMMARY

The present disclosure provides an improved fluid cartridge assembly for securely attaching and removing a sealing cap to and from a fluid cartridge.

An aspect of the present disclosure provides a cap for sealing a fluid cartridge. The cap comprises a cap body, a locking latch, first and second squeeze handles, and a lever. The locking latch extends at least partially in a proximal direction from the cap body. The locking latch is configured to engage a flange of the fluid cartridge to secure the cap to the cartridge. The first and second squeeze handles extend at least partially in a distal direction from the cap body. The first squeeze handle is operatively coupled to the locking latch and configured to be depressed in a radially inward direction toward the second squeeze handle. When the first squeeze handle is depressed in the radially inward direction the locking latch moves in a radially outward direction. The lever is coupled to at least one of the first and second squeeze handles and positioned between the first and second squeeze handles. The lever is configured to move between a first position and a second position. In the first position the first and second squeeze handles are substantially prevented from moving in the radially inward direction, and in the second position the first and second squeeze handles can move freely in the radially inward direction.

Another aspect of the present disclosure provides a fluid cartridge assembly that includes a fluid cartridge and a cap. The fluid cartridge comprises a cartridge body and a flange extending outwardly from said cartridge body. The cap is configured to couple to the fluid cartridge to seal the fluid within.

Another aspect of the present disclosure provides an alternative aspect of the fluid cartridge assembly. The fluid cartridge assembly includes a fluid cartridge and a cap. The fluid cartridge comprises a cartridge body defining a cartridge outlet at a distal end, and a flange extending outwardly from the cartridge body. The cap is configured to couple to the fluid cartridge and comprises a cap body, a plug, a locking latch, first and second squeeze handles, and a lever. The plug extends at least partially in a proximal direction from the cap body. The plug is configured to be inserted into the cartridge outlet to seal the fluid cartridge when the cap is coupled to the fluid cartridge. The locking latch extends at least partially in the proximal direction from the cap body. The locking latch is configured to engage the flange of the fluid cartridge to couple the cap to the cartridge. The first and second squeeze handles extend at least partially in a distal direction from the cap body. The first squeeze handle is operatively coupled to the locking latch and configured to be depressed in a radially inward direction toward the second squeeze handle. When the first squeeze handle is being depressed in the radially inward direction the locking latch moves in a radially outward direction. The lever is coupled to at least one of the first and second squeeze handles, and is positioned between the first and second squeeze handles. The lever is configured to move between a first position and a second position. In the first position the first and second squeeze handles are substantially prevented from moving in the radially inward direction. In the second position the first and second squeeze handles can move freely in the radially inward direction.

Another aspect of the present disclosure provides an alternative aspect of the fluid cartridge assembly. The fluid cartridge assembly includes a fluid cartridge and a cap. The fluid cartridge comprises a cartridge body defining a cartridge outlet at a distal end, and a flange extending outwardly from the cartridge body. The cap is configured to couple to the fluid multi component cartridge and comprises a cap body, multiple plugs, a locking latch, first and second squeeze handles, and a lever. The plugs extend at least partially in a proximal direction from the cap body. The plugs are configured to be inserted into the multiple cartridge outlets to seal the fluid cartridge when the cap is coupled to the fluid cartridge. The locking latch extends at least partially in the proximal direction from the cap body. The locking latch is configured to engage the flange of the fluid cartridge to couple the cap to the cartridge. The first and second squeeze handles extend at least partially in a distal direction from the cap body. The first squeeze handle is operatively coupled to the locking latch and configured to be depressed in a radially inward direction toward the second squeeze handle. When the first squeeze handle is being depressed in the radially inward direction the locking latch moves in a radially outward direction. The lever is coupled to at least one of the first and second squeeze handles, and is positioned between the first and second squeeze handles. The lever is configured to move between a first position and a second position. In the first position the first and second squeeze handles are substantially prevented from moving in the radially inward direction. In the second position the first and second squeeze handles can move freely in the radially inward direction.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown. In the drawings:

FIG. 1 illustrates a side view of a fluid cartridge assembly, according to an aspect of this disclosure.

FIG. 2 illustrates a perspective view of a distal end of a fluid cartridge, according to an aspect of this disclosure.

FIG. 3 illustrates a side view of the distal end of the fluid cartridge shown in FIG. 2, according to an aspect of this disclosure.

FIG. 4 illustrates a top perspective view of a sealing cap, according to an aspect of this disclosure.

FIG. 5 illustrates a bottom perspective view of the sealing cap shown in FIG. 4, according to an aspect of this disclosure.

FIG. 6 illustrates a first side view of the sealing cap shown in FIG. 4, according to an aspect of this disclosure.

FIG. 7 illustrates a second side view of the sealing cap shown in FIG. 4, according to an aspect of this disclosure.

FIG. 8 illustrates a top view of the sealing cap shown in FIG. 4, according to an aspect of this disclosure.

FIG. 9 illustrates a perspective view of the sealing cap shown in FIG. 4 coupled to the distal end of the fluid cartridge shown in FIG. 2, according to an aspect of this disclosure.

FIG. 10 illustrates a side view of the sealing cap shown in FIG. 4 coupled to the distal end of the fluid cartridge shown in FIG. 2, according to an aspect of this disclosure.

FIG. 11 illustrates a perspective view of a distal end of a dual fluid cartridge, according to an aspect of this disclosure.

FIG. 12 illustrates a side view of a distal end of the dual fluid cartridge shown in FIG. 11, according to an aspect of this disclosure.

FIG. 13 illustrates a top perspective view of a dual fluid sealing cap, according to an aspect of this disclosure.

FIG. 14 illustrates a bottom perspective view of the dual fluid sealing cap shown in FIG. 13, according to an aspect of this disclosure.

FIG. 15 illustrates a side view of the dual fluid sealing cap shown in FIG. 13, according to an aspect of this disclosure.

FIG. 16 illustrates a top view of the dual fluid sealing cap shown in FIG. 13, according to an aspect of this disclosure.

FIG. 17 illustrates a side view of a dual fluid cartridge assembly, according to an aspect of this disclosure.

FIG. 18 illustrates a perspective view of a distal end of an alternative aspect of a dual fluid cartridge, according to an aspect of this disclosure.

FIG. 19 illustrates a side view of a distal end of the dual fluid cartridge shown in FIG. 18, according to an aspect of this disclosure.

FIG. 20 illustrates a top perspective view of an alternative aspect of a dual cartridge sealing cap, according to an aspect of this disclosure.

FIG. 21 illustrates a bottom perspective view of the dual cartridge sealing cap shown in FIG. 20, according to an aspect of this disclosure.

FIG. 22 illustrates a first side view of the dual cartridge sealing cap shown in FIG. 20, according to an aspect of this disclosure.

FIG. 23 illustrates a second side view of the dual cartridge sealing cap shown in FIG. 20, according to an aspect of this disclosure.

FIG. 24 illustrates a top view of the dual cartridge sealing cap shown in FIG. 20, according to an aspect of this disclosure.

FIG. 25 illustrates a perspective view of the dual cartridge sealing cap shown in FIG. 20 coupled to the distal end of the dual fluid cartridge shown in FIG. 18, according to an aspect of this disclosure.

FIG. 26 illustrates a side view of the dual sealing cap shown in FIG. 20 coupled to the distal end of the dual fluid cartridge shown in FIG. 18, according to an aspect of this disclosure.

### DETAILED DESCRIPTION

The disclosure relates generally to single and dual fluid cartridge assemblies for carrying one or more fluids. The fluid cartridge assemblies includes a cap configured to mate with a fluid cartridge in such a way as to reduce the risk of accidental removal of the cap from the fluid cartridge. Accidental removal may occur during transport, child access, or other types of movement of the cartridge assembly. The cap includes a locking mechanism which substantially prevents removal of the cap from the cartridge assembly until the locking mechanism is activated.

Certain terminology is used in the description for convenience only and is not limiting. The words “proximal” and “distal” generally refer to positions or directions toward and away from, respectively, an individual operating a cartridge assembly. The words “inward”, “outward”, “axial”. “radial,” and “transverse” designate directions in the drawings to which reference is made. The term “substantially” is intended to mean considerable in extent or largely but not necessarily wholly that which is specified. The terminology includes the above-listed words, derivatives thereof and words of similar import.

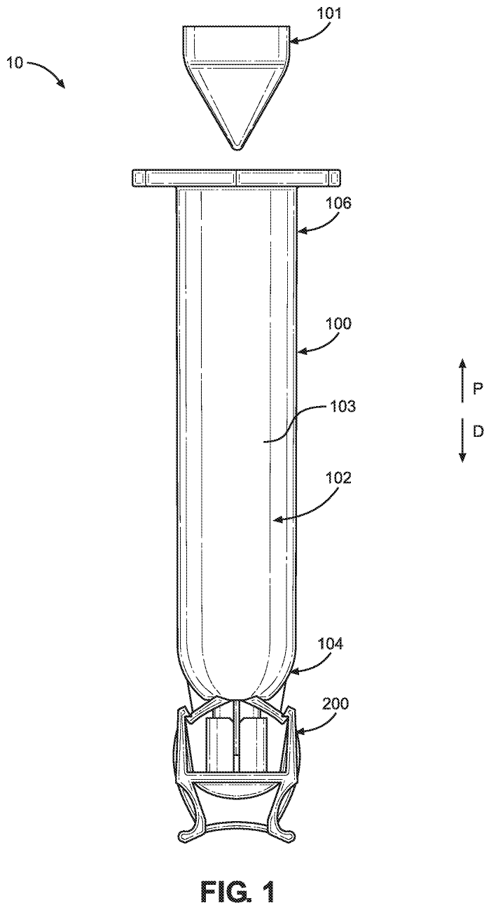

FIG. 1 illustrates a side view of a fluid cartridge assembly 10, according to an aspect of this disclosure. The fluid cartridge assembly 10 includes a fluid cartridge 100 configured to contain a fluid to be dispensed and a sealing cap 200 configured to mate with the fluid cartridge 100 to seal the fluid within the fluid cartridge 100. As illustrated in FIG. 1, the cap 200 is coupled to the fluid cartridge 100. The fluid cartridge assembly 10 may also include a piston or plunger 101 configured to slide within a fluid chamber 102 of the fluid cartridge 100. The fluid chamber 102 being defined by a body 103 of the fluid cartridge 100. To dispense the fluid, the piston 101 moves through the fluid chamber 102 in a distal direction D providing a force to the fluid that causes the fluid to dispense from a distal end 104 of the fluid cartridge 100. The piston 101 may include, for example, a pneumatically or mechanically actuated piston or other actuator configured to dispense fluid.

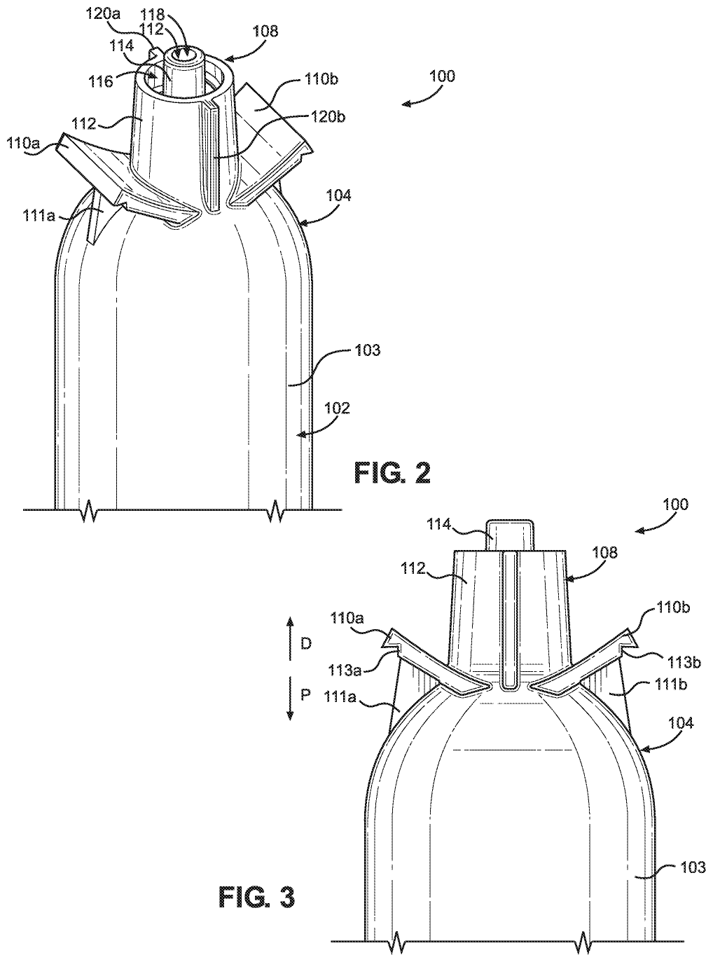

FIGS. 2 and 3 illustrate a perspective view and a side view of a distal end 104 of the fluid cartridge 100, respectively, according to aspects of this disclosure. The fluid cartridge 100 includes the fluid chamber 102 extending from the distal end 104 to a proximal end 106 of the fluid cartridge 100. In an aspect, the fluid cartridge 100 is a single fluid cartridge (e.g. 1 k cartridge). The proximal end 106 of the fluid cartridge 100 is configured to receive the piston 101 to push fluid out of the fluid chamber 102 at the distal end 104 of the fluid cartridge 100. The distal end 104 includes an outlet socket 108 for connecting to the cap 200 as described in further detail below.

The outlet socket 108 of the fluid cartridge 100 is shown in further detail in FIGS. 2 and 3. The outlet socket 108 includes a first flange 110a and a second flange 110b, a cartridge annular wall 112, and a fluid outlet wall 114. The cartridge annular wall 112 and the fluid outlet wall 114 extend distally from the distal end 104 of the fluid cartridge 100. The cartridge annular wall 112 defines a hollow port 116 within, and substantially surrounds the fluid outlet wall 114 such that the fluid outlet wall 114 is positioned within the port 116. The fluid outlet wall 114 defines a fluid outlet 118 that is in fluid communication with the fluid chamber 102. The cartridge annular wall 112 is uninterrupted about the periphery of the hollow port 116. The cartridge annular wall 112 includes a first radial projection 120a and a second radial projection 120b. The radial projections 120a and 120b extend radially outward from an outer surface of the cartridge annular wall 112. The first radial projection 120a may be spaced 180 degrees from the second radial projection 120b about the cartridge annular wall 112. In alternative aspects, there may be a single radial projection extending radially outward from the cartridge annular wall 112, or there may be more than two radial projections extending radially outward from the cartridge annular wall 112.

Each flange 110a and 110b extends outwardly from the cartridge body 103. Each flange 110a and 110b extends at least partially radial outward and at least partially in the distal direction D. In an alternative aspect, each flange 110a and 110b may extend radially outward from the cartridge body 103. In another alternative aspect, each flange 110a and 110b may extend outwardly from the cartridge annular wall 112. Each flange 110a and 110b includes a support member 111a and 111b and a locking notch 113a and 113b, respectively. Each support member 111a and 111b is attached to each respective flange 110a and 110b and to the distal end 104 of the fluid cartridge 100. Each support member 111a and 111b provides support to each respective flange 110a and 110b to minimize deflection and/or movement of each flange 110a and 110b. Each locking notch 113a and 113b is on a side of the respective flange 110a and 110b that faces at least partially in the proximal direction P. Each locking notch 113a and 113b is configured to receive corresponding structure of the cap 200 as described in further detail below.

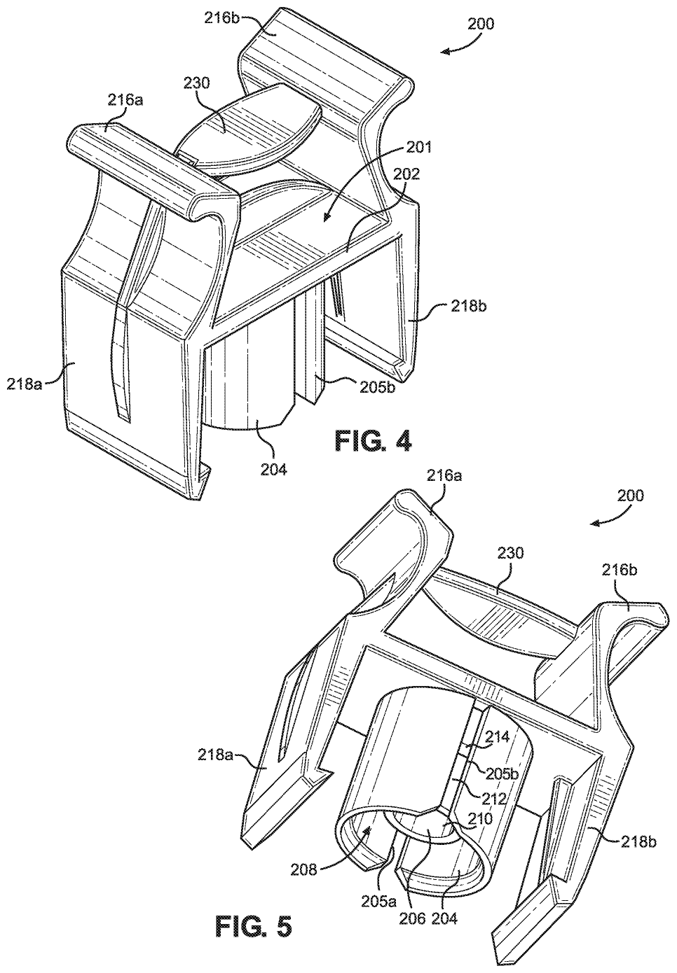

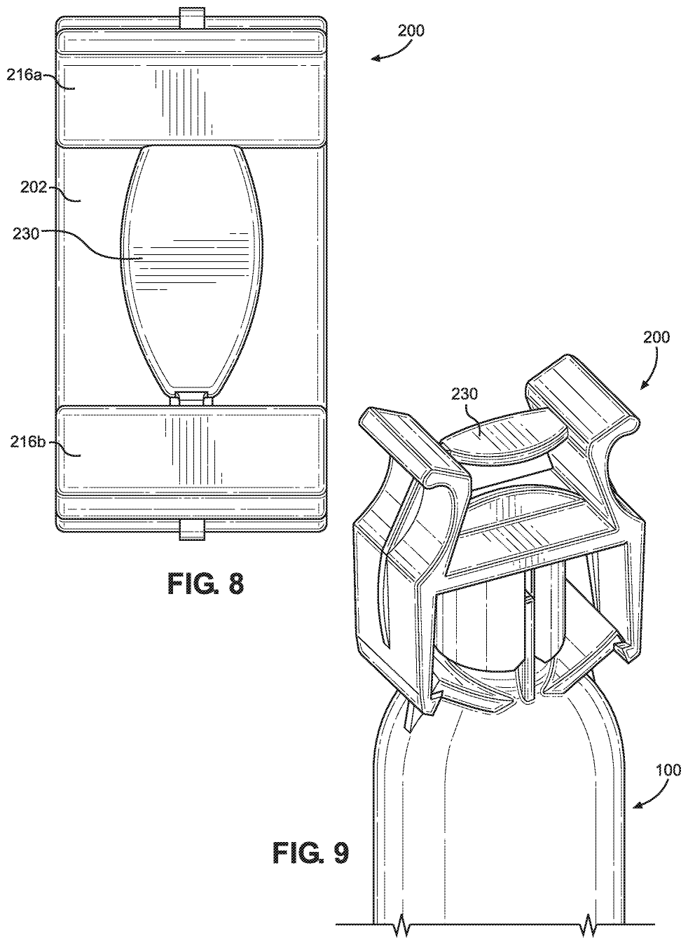

FIGS. 4 through 8 illustrate the sealing cap 200 configured to couple to the outlet socket 108 of the fluid cartridge 100, according to aspects of this disclosure. The cap 200 includes a cap body 201. The cap body 201 includes a closed distal wall 202, cap annular wall 204, and a fluid inlet wall 206. The cap annular wall 204 and the fluid inlet wall 206 extend from the closed distal wall 202 in the proximal direction P, and defines a substantially cylindrical receiving channel 208. The cap annular wall 204 includes a first slot 205a and a second slot 205b that extend through the cap annular wall 204 from a proximal end of the cap annular wall 204 towards the closed distal wall 202. In alternative aspects, there may be a single slot extending through the cap annular wall 204, or there may be more than two slots extending through the cap annular wall 204. Preferably, the number of slots extending through the cap annular wall 204 is the same as the number of radial projections extending from the cartridge annular wall 112. The cap annular wall 204 substantially surrounds the fluid inlet wall 206 such that the fluid inlet wall 206 is positioned within the receiving channel 208. The fluid inlet wall 206 has an inner surface 210 and an outer surface 212. The inner surface 210 defines a substantially cylindrical fluid channel within the fluid inlet wall 206. Alternatively, the fluid inlet wall 206 does not include the inner surface 210, and instead forms a substantially solid cylindrical member (e.g. a plug). The outer surface 212 defines a shoulder 214 that extends about a periphery of the fluid inlet wall 206.

The cap 200 further includes first and second squeeze handles 216a and 216b, first and second locking latches 218a and 218b, and a lever 230. In an aspect, the cap body 201, the first and second squeeze handles 216a and 216b, the first and second locking latches 218a and 218b, and the lever 230 are integrally molded together to form a unitary or single sealing cap 200.

The first and second squeeze handles 216a and 216b extend from the closed distal wall 202 at least partially in the distal direction D. The first squeeze handle 216a and the second squeeze handle 216b are positioned on opposing sides of the closed distal wall 202. In an aspect, the first squeeze handle 216a and the second squeeze handle 216b are substantially symmetric such that the first squeeze handle 216a is a mirror image of the second squeeze handle 216b.

The first locking latch 218a and the second locking latch 218b extend from the closed distal wall 202 at least partially in the proximal direction P. The first and second locking latches 218a and 218h are positioned radially outward from the cap annular wall 204. In an aspect, the first and second locking latches 218a and 218b extend parallel to the cap annular wall 204.

The first locking latch 218a and the second locking latch 218b include a first locking tab 222a and a second locking tab 222b, respectively. The first and second locking tabs 222a and 222h are positioned on free ends of the first and second locking latches 218a and 218b, respectively. Each locking tab 222a and 222b includes a trailing surface 224a and 224b, respectively, that extends radially inward from each respective first and second locking latch 218a and 218b. In an aspect, each trailing surface 224a and 224b extends at an oblique angle from each respective first and second locking latch 218a and 218b.

The first squeeze handle 216a is operatively coupled to the first locking latch 218a, and the second squeeze handle 216b is operatively coupled to the second locking latch 218b. The first and second squeeze handles 216a and 216b are configured to be depressed in a radially inward direction towards each other. Depressing the first and second squeeze handles 216a and 216b in the radially inward direction causes the first and second locking latches 218a and 218b to move in a radially outward direction. Conversely, releasing the first and second squeeze handles 216a and 216b from a depressed position causes the first and second locking latches 218a and 218b to move in the radially inward direction. In an aspect, the first and second locking latches 218a and 218b are biased toward the radially inward direction.

The precise appearance and structure defined by the sealing cap 200 may be modified without departing from the scope of the present disclosure. For example, the sealing cap 200 may include a single locking latch. The single locking latch may be coupled to the first squeeze handle 216a and operate in a substantially similar manner as described above. Depressing the first and second squeeze handles 216a and 216b in the radially inward direction may cause the single locking latch to move in the radially outward direction. In another alternative aspect, the first and second squeeze handles 216a and 216b may be asymmetric. For example, the first squeeze handle 216a may be coupled to the single locking latch and the second squeeze handle 216b may be sized and/or positioned at a location other than the opposing side of the closed distal wall 202 to facilitate use (e.g. gripping; depressing, etc.) by an operator.

The lever 230 is positioned between the first squeeze handle 216a and the second squeeze handle 216b, and is coupled to at least one of the first squeeze handle 216a and/or the second squeeze handle 216b. In an aspect, the lever 230 is integrally formed with or rigidly coupled to the second squeeze handle 216b, and is configured to releasably couple to the first squeeze handle 216a. For example, a tip 231 of the lever 230 may be positioned within a recess (not illustrated) formed in the first squeeze handle 216a (e.g. snap-clip). Alternatively, the tip 231 may be initially formed as part of the first squeeze handle 216a, and upon an actuation of the lever 230, the lever 230 breaks off from the first squeeze handle 216a (e.g. break-off tip). The lever 230 is positioned between the closed distal wall 202 and the distal ends of the first and second squeeze handles 216a and 216b.

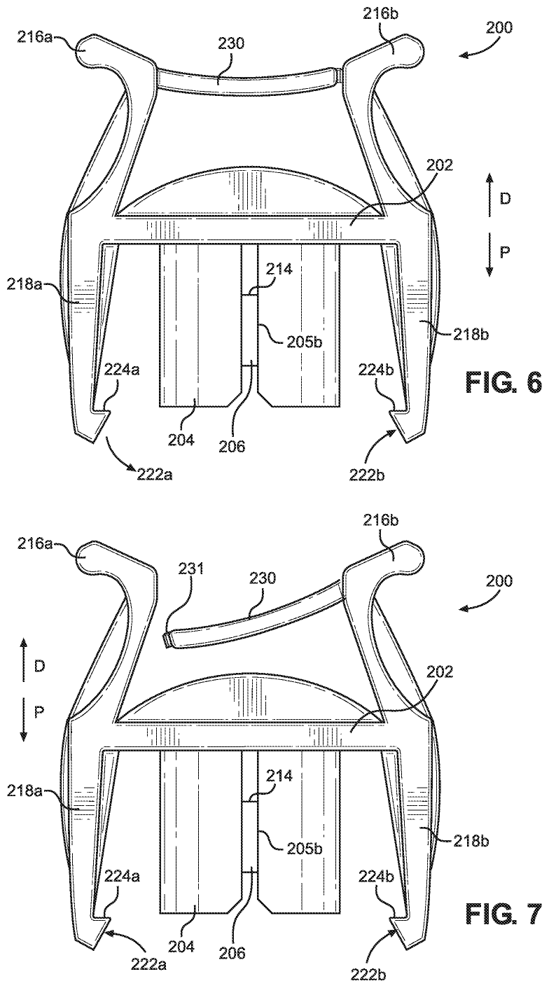

The lever 230 is configured to move between a first position and a second position. FIG. 6 illustrates the lever 230 in the first position, according to an aspect of this disclosure. In the first position, the lever 230 extends from the first squeeze handle 216a to the second squeeze handle 216h and substantially prevents movement of the first squeeze handle 216a and the second squeeze handle 216b in the radially inward direction. FIG. 7 illustrates the lever 230 in the second position, according to an aspect of this disclosure. In the second position, the first and second squeeze handles 216a and 216b can move freely in the radially inward direction.

The lever 230 is actuated between the first position and the second position by applying a force to the lever 230 in the proximal direction P. In an aspect, the lever 230 is biased toward the first position. When a force is applied by, for example, an operator, to the lever 230, the lever 230 is released and/or de-coupled from the first squeeze handle 216a and transitions from the first position to the second position.

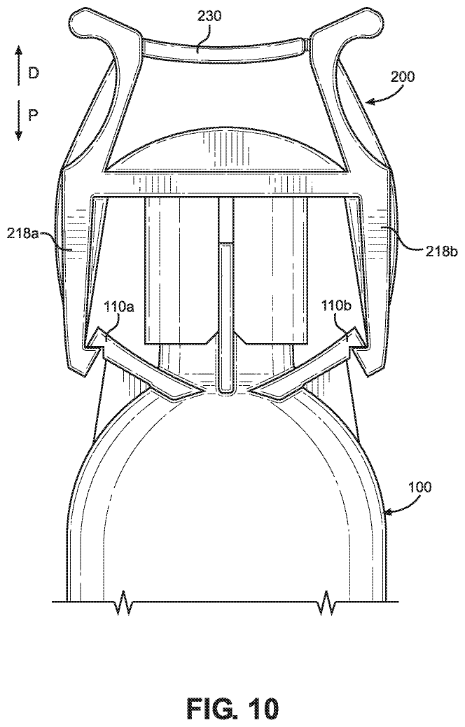

FIGS. 9 and 10 illustrate a perspective view and a side view, respectively, of the sealing cap 200 secured to the fluid cartridge 100, according to aspects of this disclosure. The first and second locking latches 218a and 218b of the sealing cap 200 are engaged with the first flange 110a and the second flange 110h of the fluid cartridge 100, respectively. The trailing surfaces 224a and 224h of each locking tab 222a and 222b abut against the locking notches 113a and 113h of the flanges 110a and 110b, respectively. The engagement between the first and second locking latches 218a and 218b and the first flange 110a and the second flange 110b secures the cap 200 to the cartridge 100, such that when the lever 230 is in the first position, removal of the cap 200 from the cartridge 100 is substantially prevented.

When the cap 200 is secured to the cartridge 100, the outlet socket 108 of the fluid cartridge 100 is positioned within the receiving channel 208 defined by the cap annular wall 204 of the cap 200. The cartridge annular wall 112 is positioned about the fluid inlet wall 206 of the cap 200. A distal end of the cartridge annular wall 112 abuts against the shoulder 214 defined by the outer surface 212 of the fluid inlet wall 206. To align the cap 200 with the cartridge 100, the radial projections 120a and 120b extending outward from the cartridge annular wall 112 are positioned within the slots 205a and 205b that extend through the cap annular wall 204. The positioning of the radial projections 120a and 120b within the slots 205a and 205h aligns the cap 200 with the fluid cartridge 100 and also prevents circumferential movement of the cap 200 relative to the fluid cartridge 100.

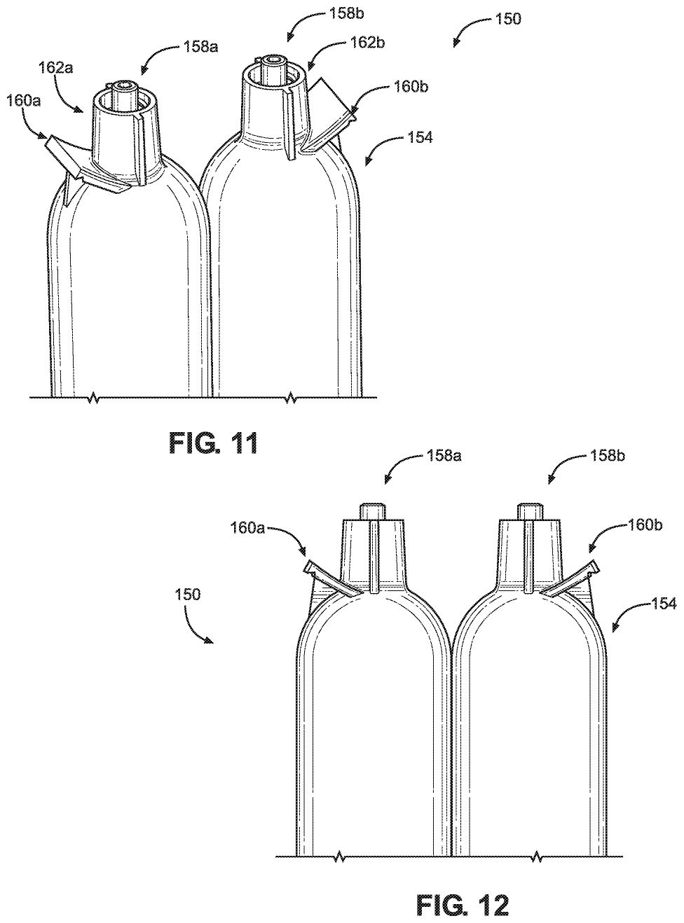

FIGS. 11 and 12 illustrate a dual fluid cartridge 150. Portions of the embodiments disclosed in FIGS. 11 and 12 are similar to aspects described above in FIGS. 2 and 3 regarding fluid cartridge 100 and those portions function similarly to those described above. The dual fluid cartridge 150 comprises a dual fluid cartridge (e.g. 2 k cartridge) configured to contain two fluids to be dispensed.

The fluid cartridge 150 includes a first fluid chamber 152a and a second fluid chamber 152b. A distal end 154 of the cartridge 150 includes a first outlet socket 158a and a second outlet socket 158b positioned on a body defining the first fluid chamber 152a and on a body defining the second fluid chamber 152b, respectively. In an aspect, the first fluid chamber 152a is configured substantially similarly to the second fluid chamber 152b. In a further aspect, the first outlet socket 158a is configured substantially similarly to the second outlet socket 158b.

The first outlet socket 158a includes a first flange 160a and a first annular wall 162a, and the second outlet socket 158b includes a second flange 160b and a second annular wall 162b. Each flange 160a and 160b may be configured in a substantially similar manner as each flange 110a and 110h of the outlet socket 108. Each flange 160a and 160h extends outwardly from the distal end 154 of the cartridge 150 at a location adjacent to the first and second outlet sockets 158a and 158b, respectively. The flanges 160a and 160b are aligned on opposing sides of the distal end 154 such that both of the first and second outlet sockets 158a and 158b are positioned between the flanges 160a and 160b.

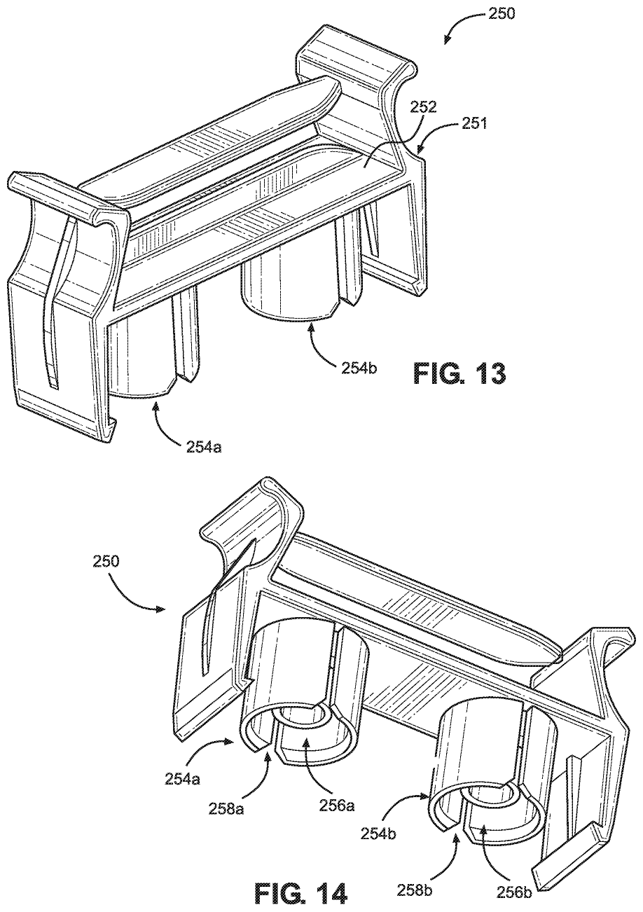

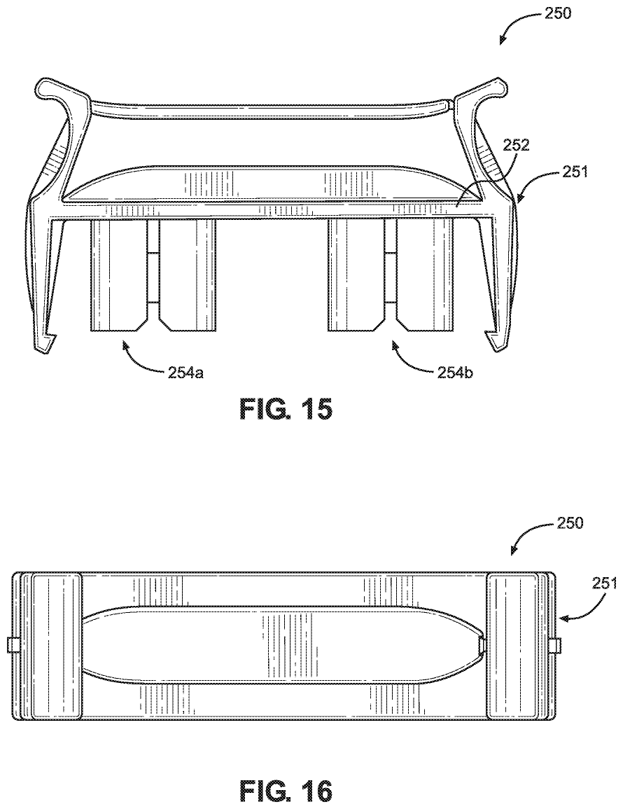

FIGS. 13 through 16 illustrate the sealing cap 250 configured to couple to the outlet sockets 158a and 158b of the fluid cartridge 150, according to aspects of this disclosure. Portions of the embodiments of the sealing cap 250 disclosed in FIGS. 13 through 16 are similar to aspects described above in FIGS. 4 through 8 regarding the sealing cap 200 and those portions function similarly to those described above.

The cap 250 includes a cap body 251. The cap body 251 includes a closed distal wall 252, a first annular wall 254a, a second annular wall 254b, a first fluid inlet wall 256a, and a second fluid inlet wall 256b. The cap annular walls 254a and 254h and the fluid inlet walls 256a and 256b extend from the closed distal wall 252. The first annular wall 254a substantially surrounds the first fluid inlet wall 256a such that the first fluid inlet wall 256a is positioned within a receiving channel 258a. The second annular wall 254b substantially surrounds the second fluid inlet wall 256b such at the second fluid inlet wall 256b is positioned within a receiving channel 258b.

When the cap 250 is secured to the fluid cartridge 150, the first outlet socket 158a of the cartridge 150 is positioned within the first receiving channel 258a defined by the first cap annular wall 254a of the cap 250, and the first fluid inlet wall 256a of the cap 250 is positioned within the first outlet socket 158a. Similarly, the second outlet socket 158b of the cartridge 150 is positioned within the second receiving channel 258b defined by the second cap annular wall 254b of the cap 250, and the second fluid inlet wall 256b of the cap 250 is positioned within the second outlet socket 158b. The first cartridge annular wall 162a and the second cartridge annular wall 162b are positioned about the first fluid inlet wall 256a and the second fluid inlet wall 256b, respectively. In an aspect, the first and second fluid inlet walls 256a and 256b form substantially solid cylindrical members (e.g. plugs). In an aspect, the first annular wall 254a and the first fluid inlet wall 256a are substantially symmetric to the second annular wall 254b and the second fluid inlet wall 256b, respectively, such that the when the cap 250 is secured to the fluid cartridge 150, the first outlet socket 158a may be positioned within the second receiving channel 258b, and the second outlet socket 158b may be positioned within the first receiving channel 258a.

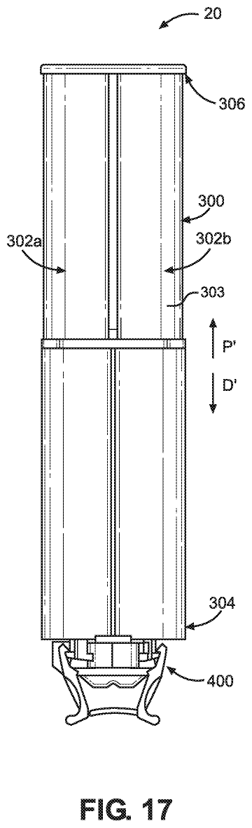

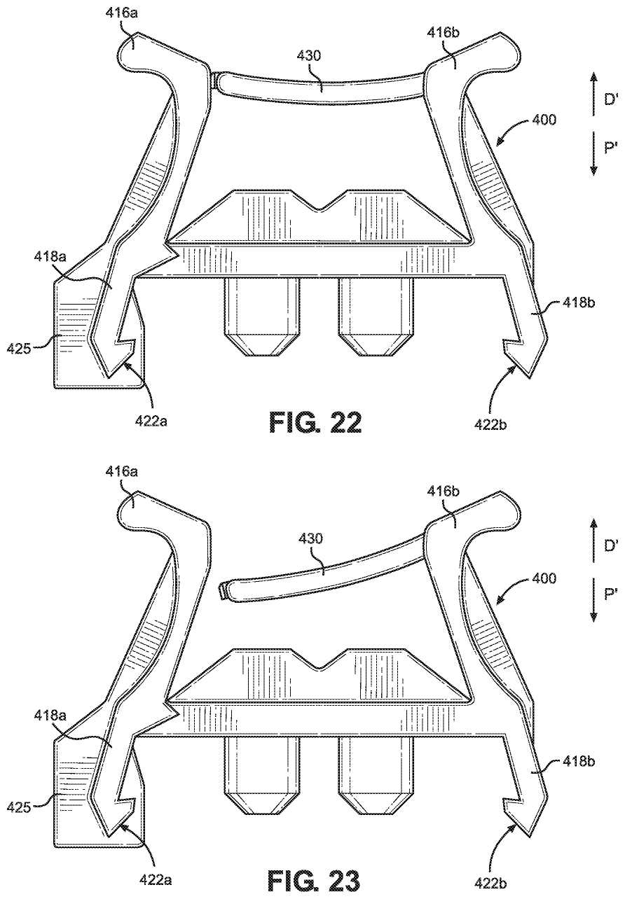

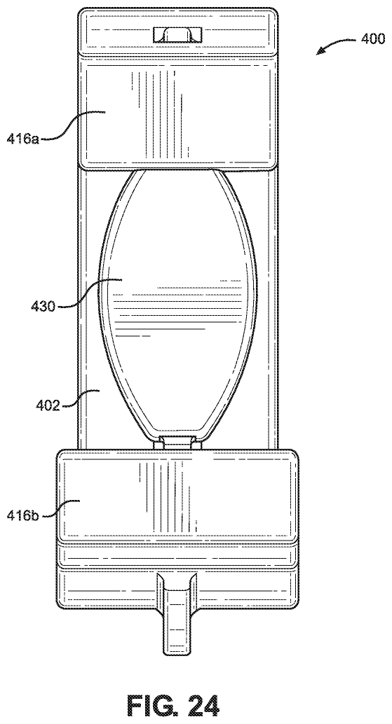

FIGS. 17 through 26 illustrate an alternate embodiment of a fluid cartridge assembly 20. Portions of the embodiment disclosed in FIGS. 17 through 26 are similar to aspects described above in FIGS. 1 through 10 and those portions function similarly to those described above. The fluid cartridge assembly 20 is a dual fluid cartridge assembly that includes a dual fluid cartridge 300 (e.g. 2 k cartridge) and a sealing cap 400. The dual fluid cartridge 300 is configured to contain two fluids to be dispensed and the sealing cap 400 is configured to mate with the dual fluid cartridge 300 to seal the fluids within the fluid cartridge 300.

The dual fluid cartridge 300 includes a first fluid chamber 302a and second fluid chamber 302b adjacent to one another for containing two fluids to be mixed together before dispensing. Although the two fluid chambers 302a and 302b are shown with similar sizes in FIG. 17, it will be appreciated that the fluid chambers 302a and 302b may be resized relative to one another in other aspects consistent with this disclosure. The fluid chambers 302a and 302b are defined by a body 303 of the fluid cartridge 300. In alternative aspects, the body 303 may include more fluid chambers without departing from this disclosure.

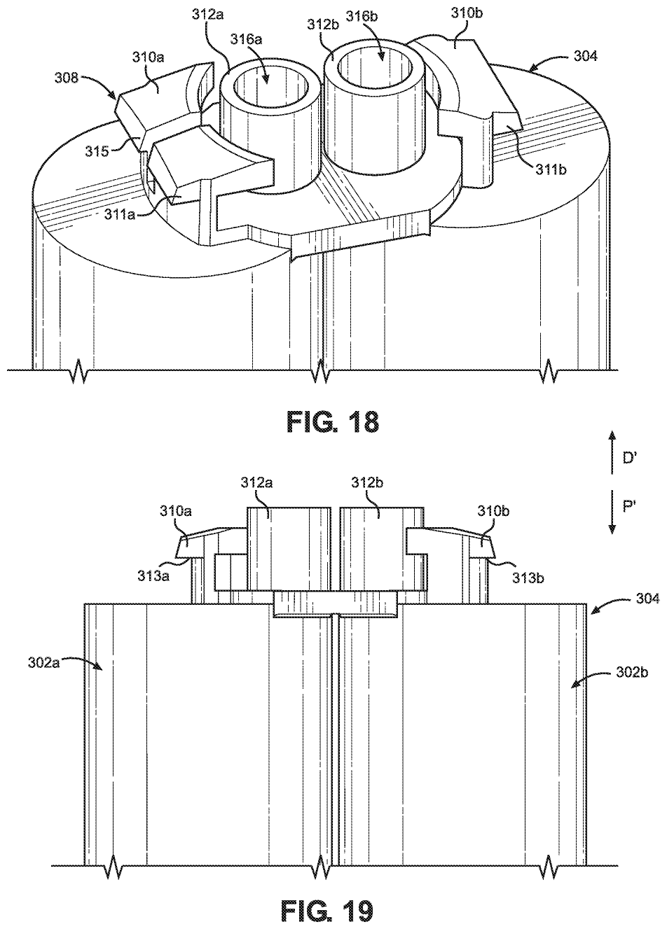

FIGS. 18 and 19 illustrate a perspective view and a side view of a distal end 304 of the fluid cartridge 300, respectively, according to aspects of this disclosure. The fluid cartridge 300 includes the fluid chambers 302a and 302b extending from the distal end 304 to a proximal end 306 of the fluid cartridge 300. The proximal end 306 of the fluid cartridge 300 is configured to receive pistons (not shown) to push fluids out of the fluid chambers 302a and 302b at the distal end 304 of the fluid cartridge 300. The distal end 304 includes an outlet socket 308 for connecting to the cap 400.

The outlet socket 308 is coupled to the body 303 of the fluid cartridge assembly 20. The outlet socket 308 includes a first flange 310a, a second flange 310b, a first cartridge annular wall 312a, and a second cartridge annular wall 312b. The first and second cartridge annular walls 312a and 312b extend distally from the distal end 304 of the fluid cartridge 300. Each cartridge annular wall 312a and 312h defines a respective hollow port 316a and 316b within. Each cartridge annular wall 312a and 312b is uninterrupted about the periphery of the hollow ports 316a and 316b, respectively.

Each flange 310a and 310b extends outwardly from the cartridge body 303. Each flange 310a and 310b extends at least partially in a distal direction D′. Each flange 310a and 310b includes and a locking notch 313a and 313b, respectively. Each locking notch 313a and 313b is on a side of the respective flange 310a and 310b that faces at least partially in a proximal direction P′. Each locking notch 313a and 313b is configured to receive corresponding structure of the cap 400 as described herein.

The first flange 310a includes a flange slot 315 that extends at least partially through the first flange 310a from a distal end towards the cartridge body 303. The flange slot 315 may be positioned at a center of the first flange 310a. In alternative aspects, the second flange 310b may also include a flange slot (not shown) extending from a distal end towards the cartridge body 303. The flange slot 315 is configured to align the fluid cartridge 300 with the sealing cap 400 as further described below.

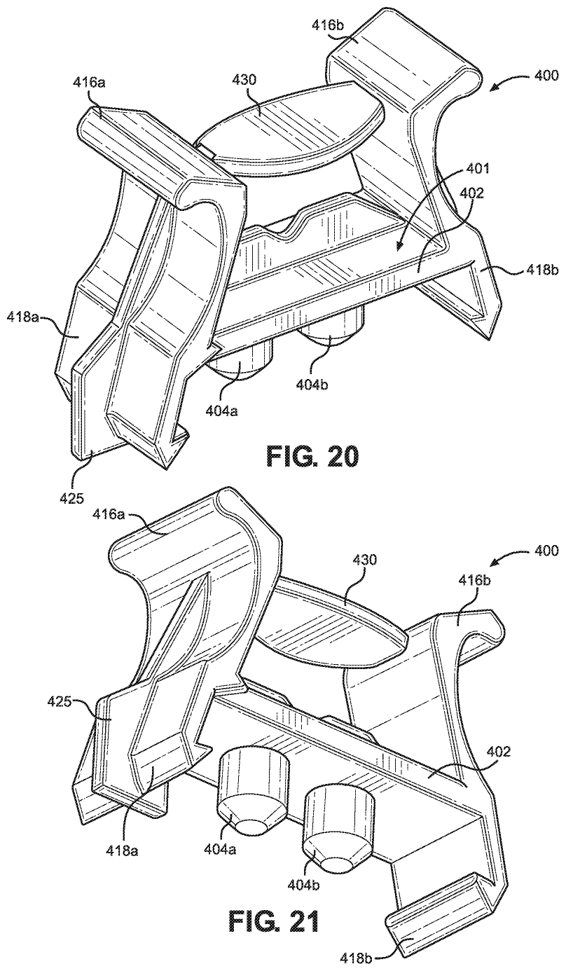

FIGS. 20 through 24 illustrate the sealing cap 400 configured to couple to the outlet socket 308 of the fluid cartridge 300, according to aspects of this disclosure. The cap 400 includes a cap body 401. The cap body 401 includes a closed distal wall 402, a first plug 404a, and a second plug 404b. The first and second plugs 404a and 404b extend from the closed distal wall 202 in the proximal direction P′. In an aspect, the first plug 404a and/or the second plug 404b may include a radial projection (not shown) configured to align the cap 400 with the outlet socket 308. In another alternative aspect, the first and second plugs 404a and 404b may be configured substantially similarly as the cap annular wall 204 and the fluid inlet wall 206 of the seal cap 200 as described above.

The cap 400 further includes first and second squeeze handles 416a and 416b, first and second locking latches 418a and 418b, and a lever 430. In an aspect, the first and second squeeze handles 416a and 416b, the first and second locking latches 418a and 418b, and the lever 430 may be configured substantially similarly as the first and second squeeze handles 216a and 216b, the first and second locking latches 218a and 218h, and the lever 230 of the seal cap 200.

The first locking latch 418a and the second locking latch 418b include a first locking tab 422a and a second locking tab 422b, respectively. The first locking tab 422a and the second locking tab 422h may be configured substantially similarly as the first and second locking tabs 222a and 222b of the seal cap 200, respectively. The first locking latch 218a may further include an orientation rib 425. The orientation rib 425 may be formed as part of the first locking latch 218a, or coupled to or attached to the first locking latch 218a. The orientation rib 425 extends at least partially radially outward from the first locking latch 218a, The orientation rib 425 may also extend beyond a proximal end of the first locking latch 218a in the proximal direction P′. The orientation rib 425 is configured to fit within the flange slot 315 of the first flange 310a to align the seal cap 400 with the fluid cartridge 300. The extension of the orientation rib 425 beyond the proximal end of the first locking latch 218a may help orient the cap 400 when the cap 400 is being attached to the fluid cartridge 300. It will be appreciated that the second locking latch 218b may also include an orientation rib (not shown) configured substantially similarly as the orientation rib 425 of the first locking latch 218a.

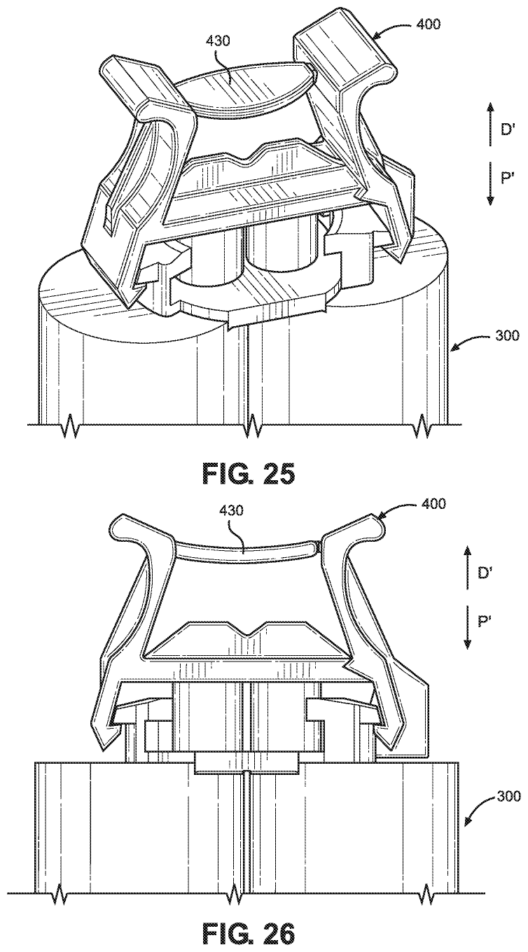

FIGS. 25 and 26 illustrate a perspective view and a side view, respectively, of the sealing cap 400 secured to the fluid cartridge 300, according to aspects of this disclosure. The first and second locking latches 418a and 418h of the sealing cap 400 are engaged with the first flange 310a and the second flange 310b of the fluid cartridge 300, respectively. The engagement between the first and second locking latches 418a and 418b and the first flange 310a and the second flange 310b may be substantially similar to the engagement between the first and second locking latches 218a and 218h of the seal cap 200 and the first flange 110a and the second flange 110b of the cartridge 100, as described above.

When the cap 400 is attached to the fluid cartridge 300, the plugs 404a and 404b of the cap 400 are positioned within the hollow ports 316a and the 316b defined by the first and second cartridge annular walls 312a and 312b of the outlet socket 308. The cartridge annular wall 112 is positioned about the fluid inlet wall 206 of the cap 200. A distal end of the cartridge annular wall 112 abuts against the closed distal wall 402 of the cap 400. To align the cap 400 with the fluid cartridge 300, the orientation rib 425 extending outward from the first locking latch 418a is positioned within flange slot 315 that extends through the first flange 310a. The positioning of the orientation rib 425 within the flange slot 315 aligns the cap 400 with the fluid cartridge 300 and also prevents circumferential movement of the cap 400 relative to the fluid cartridge 300.

It will be appreciated that the elements described with respect to the fluid cartridge assembly 20 may be incorporated into the fluid cartridge assembly 10, and similarly, the elements described with respect to the fluid cartridge assembly 10 may be incorporated into the fluid cartridge assembly 20.

One example of a method for using the fluid cartridge assembly 10 commences by attaching the seal cap 200 to the fluid cartridge 100. The cap 200 is attached to the fluid cartridge by inserting the cartridge annular wall 112 of the fluid cartridge 100 within the cap annular wall 204 of the cap 200. As the cartridge annular wall 112 is being inserted, the radial projections 120a and 120h slide within the slots 205a and 205h defined by the cap annular wall 204, respectively, maintaining alignment of the cap 200 relative to the cartridge 100. After the cartridge annular wall 112 is inserted, the locking tabs 222a and 222b of the locking latches 218a and 218b engage the first and second flanges 110a and 110b of the outlet socket 108 securing the cap 200 to the cartridge 100.

During attachment of the cap 200 to the cartridge 100, the lever 230 may either be in the first position, or may be depressed into the second position. For example, if the lever 230 is in the first position, the locking latches 218a and 218b may flex radially outward as the locking tabs 222a and 222h slide over and engage the first and second flanges 110a and 110b. Alternatively, the locking latches 218a and 218b may rigid, such that the lever must be depressed and transitioned to the second position to enable the locking latches 218a and 218b to move radially outward so that the locking tabs 222a and 222b may slide over and engage the first and second flanges 110a and 110b. After the locking tabs 222a and 222b are engaged with the first and second flanges 110a and 110b, the lever 230 transitions from the second position to the first position.

To remove the cap 200 from the cartridge 100, an operator may transition the lever 230 from the first position to the second position by depressing the lever 230 in the proximal direction P. Once the lever 230 is in the second position, the squeeze handles 216a and 216b are free to move in the radially inward direction. The operator may squeeze the handles 216a and 216b to move them radially inward, which causes the locking latches 218a and 218b to move radially outward disengaging the locking tabs 222a and 222b from the first and second flanges 110a and 110b. The cap 200 may be moved in the distal direction D away from the cartridge 100, thereby removing the cap 200.

Although reference was made to the fluid cartridge assembly 10 in the above described example for using the fluid cartridge assembly 10, similar methods may also be employed by the fluid cartridge assembly 20.

It will be appreciated that the foregoing description provides examples of the disclosed system and method. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

What is claimed is:

1. A cap for sealing a cartridge containing fluid, the cap comprising:
a cap body;
a locking latch extending at least partially in a proximal direction from said cap body, said locking latch configured to engage a flange of the cartridge to secure the cap to the cartridge;
first and second squeeze handles extending at least partially in a distal direction from said cap body, said first squeeze handle being operatively coupled to said locking latch and configured to be depressed in a radially inward direction toward said second squeeze handle, wherein when said first squeeze handle is depressed in the radially inward direction said locking latch moves in a radially outward direction; and
a lever coupled to at least one of said first and second squeeze handles and positioned between said first and second squeeze handles, said lever configured to move between a first position and a second position, wherein in the first position said first and second squeeze handles are substantially prevented from moving in the radially inward direction, and wherein in the second position said first and second squeeze handles can move freely in the radially inward direction.

2. The cap of claim 1, wherein e locking latch is a first locking latch, the cap further comprising:
a second locking latch extending at least partially in the proximal direction from said cap body, said second locking latch configured to engage a second flange of the cartridge to further secure the cap to the cartridge, second locking latch being positioned on an opposing side of said cap body from said first locking latch,
wherein said second squeeze handle is operatively coupled to said second locking latch and configured to be depressed in a radially inward direction toward said first squeeze handle, wherein when said second squeeze handle is depressed in the radially inward direction said second locking latch moves in a radially outward direction.

3. The cap of claim 1, wherein said cap body comprises a closed distal wall and an annular wall, said locking latch and said first and second squeeze handles being coupled to the closed distal wall, the annular wall extending from the closed distal wall in the proximal direction, the annular wall defining a substantially cylindrical receiving channel.

4. The cap of claim 3, wherein the annular wall includes at least one slot that extends through the annular wall from a proximal end of the annular wall towards the closed distal wall.

5. The cap of claim 3, wherein the cap body further comprises a fluid inlet wall, the fluid inlet wall extending from the closed distal wall in the proximal direction, the fluid inlet wall being positioned within the receiving channel, the fluid inlet wall having an inner surface that defines a substantially cylindrical fluid channel within and an outer surface that defines a shoulder thereon.

6. The cap of claim 3, wherein the annular wall is a first annular wall and the receiving channel is a first receiving channel, wherein said cap body further comprises a second annular wall extending from the closed distal wall in the proximal direction, the second annular wall defining a second receiving channel that is substantially cylindrical.

7. The cap of claim 1, wherein said cap body, said locking latch, said first and second squeeze handles, and said lever are molded together forming a unitary cap.

8. The cap of claim 1, wherein said lever is rigidly coupled to said first squeeze handle and releasably coupled to said second squeeze handle, wherein a force applied to said lever in the proximal direction transitions said lever from the first position to the second position such that said lever is de-coupled from said second squeeze handle.

9. The cap of claim 1, wherein said locking latch comprises an orientation rib attached thereto, the orientation rib extending at least partially radially outward from said locking latch.

10. The cap of claim 8, wherein a proximal end of said orientation rib extends beyond a proximal end of said locking latch in the proximal direction.

11. A fluid cartridge assembly comprising:
a fluid cartridge comprising:
a cartridge body, and
a flange extending outwardly from said cartridge body; and
a cap configured to couple to said fluid cartridge, the cap comprising:
a cap body,
a locking latch extending at least partially in a proximal direction from said cap body, said locking latch configured to engage said flange of said fluid cartridge to couple said cap to said cartridge,
first and second squeeze handles extending at least partially in a distal direction from said cap body, said first squeeze handle being operatively coupled to said locking latch and configured to be depressed in a radially inward direction toward said second squeeze handle, wherein when said first squeeze handle is depressed in the radially inward direction said locking latch moves in a radially outward direction, and
a lever coupled to at least one of said first and second squeeze handles and positioned between said first and second squeeze handles, said lever configured to move between a first position and a second position, wherein in the first position said first and second squeeze handles are substantially prevented from moving in the radially inward direction, and wherein in the second position said first and second squeeze handles can move freely in the radially inward direction.

12. The assembly of claim 11, wherein said cap body comprises a closed distal wall and a cap annular wall, said locking latch and said first and second squeeze handles being coupled to the closed distal wall, the cap annular wall extending from the closed distal wall in the proximal direction, the cap annular wall defining a substantially cylindrical receiving channel, and
wherein said fluid cartridge further comprises a cartridge annular wall extending from a distal end of said cartridge body in the distal direction, the cartridge annular wall configured to be received within the cap annular wall.

13. The assembly of claim 12, wherein the cap annular wall includes at least one slot that extends through the cap annular wall from a proximal end of the cap annular wall towards the closed distal wall, and wherein the cartridge annular wall includes at least one radial projection, wherein each of the at least one radial projections corresponds to a respective at least one slot of said cap annular wall such that when said cap is coupled to said fluid cartridge each at least one radial projection is positioned at least partially within a respective at least one slot.

14. The assembly of claim 12, wherein said cap body further comprises a fluid inlet wall, the fluid inlet wall extending from the closed distal wall in the proximal direction, the fluid inlet wall being positioned within the receiving channel, the fluid inlet wall having an inner surface that defines a substantially cylindrical fluid channel within and an outer surface that defines a shoulder thereon, and
wherein said fluid cartridge further comprises a fluid outlet wall, the fluid outlet wall extending from the distal end of the said cartridge body in the distal direction, the fluid outlet wall configured to be received within the receiving channel of the annular cap wall and about the fluid inlet wall, wherein when said cap is coupled to said fluid cartridge a distal end of the fluid outlet wall contacts the shoulder of the fluid inlet wall.

15. The assembly of claim 11, wherein said locking latch comprises an orientation rib, the orientation rib extending at least partially radially outward from said locking latch, and
wherein said flange defines an orientation slot, wherein when said cap is coupled to said fluid cartridge the orientation rib is positioned at least partially within the orientation slot.

16. The assembly of claim 11, wherein said lever is rigidly coupled to said first squeeze handle and releasably coupled to said second squeeze handle, wherein a force applied to said lever in the proximal direction transitions said lever from the first position to the second position such that said lever is de-coupled from said second squeeze handle.

17. A fluid cartridge assembly comprising:
a fluid cartridge comprising:
a cartridge body defining a cartridge outlet at a distal end, and
a flange extending outwardly from said cartridge body; and
a cap configured to couple to said fluid cartridge, the cap comprising:
a cap body,
a plug extending at least partially in a proximal direction from said cap body, the plug configured to insert into the cartridge outlet to seal said fluid cartridge when the cap is coupled to the fluid cartridge,
a locking latch extending at least partially in the proximal direction from said cap body, said locking latch configured to engage said flange of said fluid cartridge to couple said cap to said cartridge,
first and second squeeze handles extending at least partially in a distal direction from said cap body, said first squeeze handle being operatively coupled to said locking latch and configured to be depressed in a radially inward direction toward said second squeeze handle, wherein when said first squeeze handle is depressed in the radially inward direction said locking latch moves in a radially outward direction, and
a lever coupled to at least one of said first and second squeeze handles and positioned between said first and second squeeze handles, said lever configured to move between a first position and a second position, wherein in the first position said first and second squeeze handles are substantially prevented from moving in the radially inward direction, and wherein in the second position said first and second squeeze handles can move freely in the radially inward direction.

18. The assembly of claim 17, wherein the cartridge outlet defined by said cartridge body is a first cartridge outlet, and wherein said plug is a first plug, the fluid cartridge assembly further comprising:
a second cartridge outlet defined by said cartridge body at the distal end; and
a second plug extending at least partially in a proximal direction from said cap body, said second plug configured to insert into said second cartridge outlet to seal said fluid cartridge when said cap is coupled to said fluid cartridge.

19. The assembly of claim 18, wherein said locking latch is a first locking latch, and wherein said flange is a first flange, said fluid cartridge further comprising:
a second flange extending outwardly from said cartridge body; and
said cap further comprising:
a second locking latch extending at least partially in the proximal direction from said cap body, said second locking latch configured to engage said second flange of said fluid cartridge to couple said cap to said cartridge,
wherein said second squeeze handle is operatively coupled to said second locking latch and configured to be depressed in a radially inward direction toward said first squeeze handle, wherein when said second squeeze handle is depressed in the radially inward direction said second locking latch moves in a radially outward direction.

20. The assembly of claim 17, wherein said locking latch comprises an orientation rib attached thereto, the orientation rib extending at least partially radially outward from said locking latch, and wherein said flange includes a flange slot that extends at least partially through said flange from a distal end of said flange to said cartridge body, the orientation rib being configured to fit within the flange slot to align said cap with said fluid cartridge.

21. A fluid cartridge assembly comprising:
a multi-component fluid cartridge comprising:
a cartridge body defining a first chamber and a second chamber,
a first outlet socket in fluid communication with the first chamber,
a second outlet socket in fluid communication with the second chamber, and
a flange extending outwardly from said cartridge body; and
a cap configured to couple to said fluid cartridge, the cap comprising:
a cap body,
a first plug configured to be positioned within the first outlet socket,
a second plug configured to be positioned within the second outlet socket,
a locking latch extending at least partially in a proximal direction from said cap body, said locking latch configured to engage said flange of said fluid cartridge to couple said cap to said cartridge,
first and second squeeze handles extending at least partially in a distal direction from said cap body, said first squeeze handle being operatively coupled to said locking latch and configured to be depressed in a radially inward direction toward said second squeeze handle, wherein when said first squeeze handle is depressed in the radially inward direction said locking latch moves in a radially outward direction, and
a lever coupled to at least one of said first and second squeeze handles and positioned between said first and second squeeze handles, said lever configured to move between a first position and a second position, wherein in the first position said first and second squeeze handles are substantially prevented from moving in the radially inward direction, and wherein in the second position said first and second squeeze handles can move freely in the radially inward direction.

22. The assembly of claim 21, wherein the locking latch is a first locking latch and the flange is a first flange, the cap further comprising a second locking latch extending at least partially in the proximal direction from said cap body, the multi-component fluid cartridge further comprising a second flange, wherein said second locking latch is configured to engage said second flange of the cartridge to further secure the cap to the cartridge, said second locking latch being positioned on an opposing side of said cap body from said first locking latch,
wherein said second squeeze handle is operatively coupled to said second locking latch and configured to be depressed in a radially inward direction toward said first squeeze handle, wherein when said second squeeze handle is depressed in the radially inward direction said second locking latch moves in a radially outward direction.

23. The assembly of claim 21, wherein said cap body comprises a closed distal wall, wherein said locking latch and said first and second squeeze handles are coupled to the closed distal wall, and wherein said first plug extends from the closed distal wall in the proximal direction and said second plug extends from the closed distal wall in the proximal direction.

24. The assembly of claim 23, wherein the cap body further comprises a first annular wall extending from the closed distal wall in the proximal direction and a second annular wall extending from the closed distal wall in the proximal direction, said first annular wall being configured to receive the first outlet socket within, and said second annular wall being configured to receive the second outlet socket within.
